(19) United States
(12) Patent Application Publication
(10) Pub. No.: US 2024/0408324 A1
(43) Pub. Date: Dec. 12, 2024

(54) DEVICE FOR DISPENSING A FLUID PRODUCT, AND METHOD FOR PRIMING SAME

(71) Applicant: APTAR FRANCE SAS, Le Neubourg (FR)

(72) Inventor: Matthieu SAVALLE, Oissel (FR)

(73) Assignee: APTAR FRANCE SAS, Le Neubourg (FR)

(21) Appl. No.: 18/719,030

(22) PCT Filed: Dec. 9, 2022
(86) PCT No.: PCT/FR2022/052301

§ 371 Date: Jun. 12, 2024

(30) Foreign Application Priority Data
Dec. 13, 2021 (FR) 2113413

(51) Int. Cl. A61M 11/00; A61M 15/08

(52) U.S. Cl. CPC A61M 11/007; A61M 15/08; A61M 2205/073

(57) ABSTRACT

Fluid product device having a dispenser joined to a reservoir containing fluid product, the dispenser having a pump body containing a dosing chamber, and a dispensing head with a dispensing aperture, a plunger secured to the dispensing head to slide in the dosing chamber between rest and actuation positions. The dosing chamber has at least two openings, a first opening for filling the dosing chamber and a second opening connecting the dosing chamber to the dispensing aperture. The first opening is made in a cylindrical portion of the pump body in which said plunger slides during actuation, the first opening connecting the dosing chamber to the reservoir when the plunger is in the rest position and isolating the dosing chamber from the reservoir when the plunger moves out of its rest position. The dosing chamber is filled through the first opening when the dispenser is joined to the reservoir.

the present invention relates to a device for dispensing a fluid product, in particular for a nasal delivery of one or two doses of a pharmaceutical fluid product. The present invention also relates to a method for priming a device for dispensing a fluid product.

A disadvantage of certain devices for a nasal delivery of fluid product, in particular those using a pump with a dosing chamber to define the volume of the dose dispensed upon each actuation, relates to the obligation to prime the pump to replace the air contained initially in the dosing chamber with fluid. This priming is generally done by one or more actuations of the device, consequently with a possible loss of fluid product.

The present invention aims to provide a device for dispensing a fluid product and a method for priming same, which do not have the abovementioned disadvantages.

More specifically, the present invention aims to provide such a device and method which require no priming of the pump.

The present invention also aims to provide such a device and method which are simple and inexpensive to manufacture and to assemble.

The present invention therefore aims for a fluid product device comprising a dispenser joined to a reservoir which contains fluid product, said dispenser comprising a pump body which contains a dosing chamber, and a dispensing head comprising a dispensing aperture, said dispensing head being axially movable with respect to said pump body between a rest position and an actuation position, a plunger, which is secured to said dispensing head, being designed to slide in said dosing chamber between a rest position and an actuation position, said dosing chamber comprising at least two openings, a first opening for filling said dosing chamber and a second opening connecting said dosing chamber to said dispensing aperture, said first opening being made in a cylindrical portion of said pump body, in which said plunger slides during actuation, said first opening connecting said dosing chamber to said reservoir when said plunger is in the rest position, and said first opening isolating said dosing chamber from said reservoir when said plunger moves out of its rest position, said dosing chamber being filled through said first opening when said dispenser is joined to said reservoir.

Advantageously, a lower edge of said first opening is disposed at rest at a distance d1 from said plunger, such that during actuation, said first opening is closed after an initial stroke corresponding to said distance d1.

Advantageously, said distance d1 forms less than 30% of the total actuation stroke of said plunger, preferably around 20%.

Advantageously, said first opening comprises one or more slots formed in the cylindrical portion of said pump body.

Advantageously, a hollow insert is disposed in said dispensing head, said hollow insert defining an expulsion channel connecting, during actuation, said dosing chamber to said dispensing aperture, said hollow insert comprising said plunger.

Advantageously, said plunger is formed by a seal, in particular an O-ring, joined to said hollow insert.

According to a first advantageous embodiment, said reservoir only contains a single dose of fluid product, dispensed in a single actuation.

According to a second advantageous embodiment, said reservoir contains several doses of fluid product dispensed in several successive actuations.

Advantageously, said reservoir contains two doses of fluid product dispensed in two successive actuations.

Advantageously, said dosing chamber comprises a third opening provided at the lower axial end of said pump body, said third opening comprising a stopper member, such as a ball, forming an inlet valve for said dosing chamber for filling it after each actuation.

The present invention also aims for a method for priming a device for dispensing fluid product, comprising the following steps:
providing a reservoir which contains at least one dose of fluid product;
providing a dispenser comprising a pump body which contains a dosing chamber, and a dispensing head comprising a dispensing aperture, said dispensing head being axially movable with respect to said pump body between a rest position and an actuation position, a plunger, which is secured to said dispensing head, being designed to slide in said dosing chamber between a rest position and an actuation position, said dosing chamber comprising at least two openings, a first opening for filling said dosing chamber and a second opening connecting said dosing chamber to said dispensing aperture, said first opening being made in a cylindrical portion of said pump body in which said plunger slides during actuation, said first opening connecting said dosing chamber to said reservoir when said plunger is in the rest position, and said first opening isolating said dosing chamber from said reservoir when
said plunger moves out of its rest position, said method comprising the step of joining said dispenser to said reservoir, by inserting at least partially said pump body in said reservoir with said plunger in the rest position, this insertion moving the fluid product contained in said reservoir, said moved fluid product penetrating inside said dosing chamber through said first opening, to thus prime the device.

These features and advantages and others of the present invention will appear more clearly during the following detailed description, made in reference to the accompanying drawings, given as non-limiting examples, and in which:

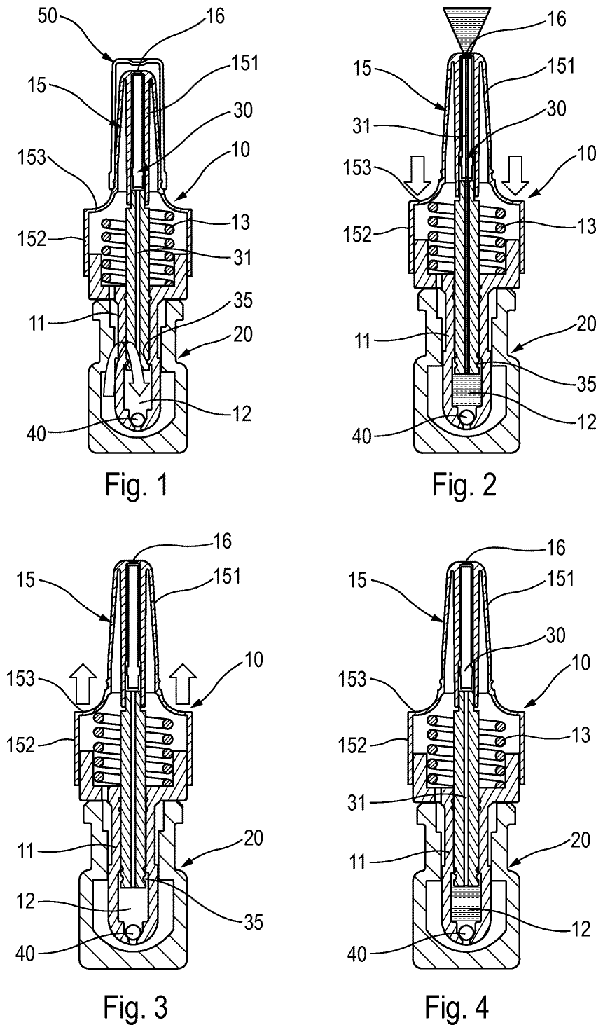

FIG. 1 is a schematic, cross-sectional view of a fluid product dispensing device according to an advantageous embodiment, during the joining of the dispenser to the reservoir,

FIG. 2 is a view similar to that of FIG. 1, during the first actuation,

FIG. 3 is a view similar to that of FIG. 2, after the first actuation,

FIG. 4 is a view similar to that of FIG. 3, after loading the dosing chamber with another dose of fluid product;

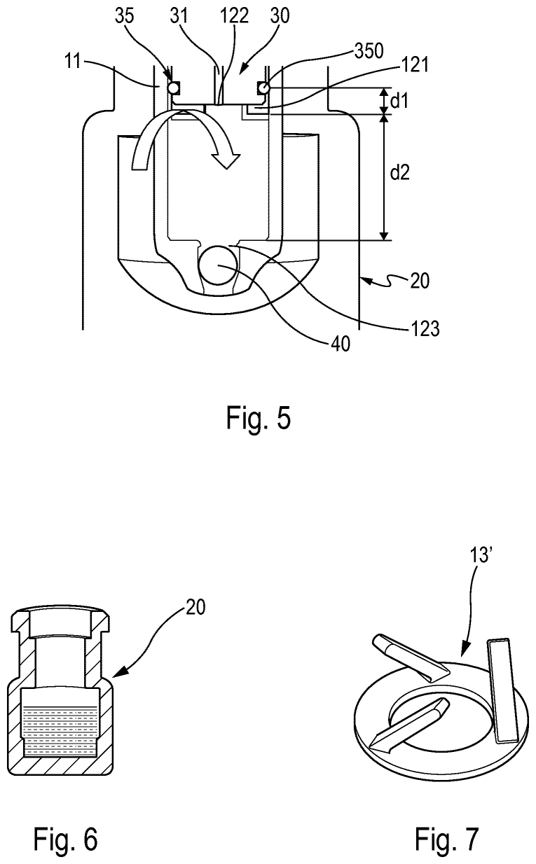

FIG. 5 is an enlarged, schematic view of a portion of FIG. 1;

FIG. 6 is a schematic, cross-sectional view of a reservoir before joining of the dispenser, and

FIG. 7 is a schematic, perspective view of a plastic spring being able to replace the metal spring of FIGS. 1 to 4.

The terms “axial” and “radial” are relative to the longitudinal central axis of the device. The terms “upstream” and “downstream” are relative to the direction of flow of the fluid during actuation. The terms “top” and “bottom” refer to the upright position of the device represented in FIGS. 1 to 4.

In the advantageous embodiment of FIGS. 1 to 5, a dispenser 10 is joined to a reservoir 20 which contains the fluid product to be dispensed, typically a pharmaceutical-type liquid.

The reservoir 20 is advantageously formed by a hollow and blind body, comprising a single opening and which contains one or more dose(s) of fluid product to be dispensed. An example of an implementation of this reservoir 20 designed for the embodiment of FIGS. 1 to 5 is represented in an isolated manner in FIG. 6.

The present invention applies to a single-dose device which only contains a single dose of fluid product in the reservoir 20, but it is particularly designed for so-called two-dose devices, where the reservoir 20 contains two doses of fluid product, dispensed in two successive actuations. Optionally, the reservoir 20 could contain more than two doses, for example three or four doses.

The dispenser 10 comprises a pump body 11 which contains a dosing chamber 12, and a dispensing head 15 comprising a dispensing aperture 16, preferably oriented axially. The dispensing orifice 16 serves to dispense a dose of fluid out from said dispensing head 15 while the device is being actuated by a user. The dispensing head 15 is axially movable with respect to the pump body 11 between a rest position and an actuation position.

At rest, a removable protective cap 50 can be disposed on the dispensing head 15 to protect the dispensing aperture 16, as can be seen in FIG. 1.

A spring 13 is disposed between the pump body 11 and the dispensing head 15 to urge the latter to its rest position.

In the embodiment of FIGS. 1 to 4, the spring 13 is of the conventional coil type, generally metal.

In a variant, it can be considered to replace the spring 13 with a plastic elastically deformable element 13′. FIG. 7 shows an example of such an elastic element 13′.

The dispensing head 15 comprises an extended nasal endpiece 151 comprising, at its axial end, said dispensing aperture 16, as well as a side body 152, connected to said nasal endpiece 151 via an almost radial flange 153.

A hollow insert 30 is disposed in the dispensing head 15, said hollow insert 30 defining an expulsion channel 31.

Advantageously, spray profile can be provided directly upstream of said dispensing aperture 16, between said insert 30 and the bottom wall of the dispensing head 15.

On the opposite side, the hollow insert 30 comprises a plunger 35 designed to slide in the dosing chamber 12 of the pump body 11 between a rest position and an actuation position. In the example of the figures, a gasket 350, in particular an O-ring, is assembled on the hollow insert 30 in order to form the piston, as can be seen more particularly in FIG. 5. In a variant, the plunger 35 could be formed integrally with the hollow insert 30.

The joining of the dispenser 10 to the reservoir 20 can be done in any known manner, for example by using a fixing ring to fix the pump body 11 to a neck of the reservoir.

According to the invention, the dosing chamber 12 comprises at least two openings 121, 122, 123. These openings are distinct, and each have a specific function.

In the case of a single dose, when the reservoir 20 only contains a single dose of fluid product, there are two openings 121 and 122.

In the case of a two-dose or multidose device, when the reservoir 20 contains at least two doses of fluid product, there are three openings 121, 122 and 123.

The first opening 121 is made in the cylindrical portion of the pump body 11 in which the plunger 35 slides during actuation.

This first opening 121 connects the dosing chamber 12 to the reservoir 20 when the plunger 35 is in the rest position. During actuation, the piston 35 slides axially downwards in the pump body 11. Optionally, in particular in the case of a two-dose, after dispensing of the first dose, the plunger 35 raises axially to return into its rest position. At the end of this return stroke, the first opening 121 is opened again.

As can be seen in FIG. 5, the first opening 121 is preferably disposed at rest in the proximity of the plunger 35, such that after a initial small actuation stroke d1, illustrated in FIG. 5, the plunger 35 extends beyond the first opening 121, and for the rest of the actuation stroke d2, this first opening 121 is therefore closed. In other words, a lower edge of the first opening 121 is disposed at rest at a distance d1 from the plunger 35, such that, during actuation, said first opening 121 is closed after an initial stroke corresponding to said distance d1. Advantageously, the distance d1 forms less than 30% of the total actuation stroke d1+d2 of the plunger 35, preferably around 20%.

Advantageously, the first opening 121 comprises one or more slots formed in the cylindrical portion of the pump body 11. The number of slots, which could also be holes, can be any number, for example one, two, three or four slots.

The second opening 122 is axial and is provided at the lower axial end of the hollow insert 30. The second opening 122 thus connects the dosing chamber 12 to the expulsion channel 31.

The third opening 123 is axial and is provided at the lower axial end of the pump body 11. This third opening is only provided if the device is a two-dose or a multidose device. A stopper member 40, such as a ball, is provided to close the third opening 123 from the start of actuation, and to open the third opening 123 from the start of the return stroke of the plunger 35, when it returns from its actuation position to its rest position. This third opening 123 together with the stopper member 40 therefore forms a conventional inlet valve for the dosing chamber 12, for filling it after each actuation of the pump due to the depression generated by the plunger 35 which raises to its rest position.

In the example represented in the figures, the dosing chamber 12 does not comprise any outlet valve at the second opening 122, and the dispensing aperture 16 does not comprise any stopper. The dosing chamber 12 is therefore permanently connected to the atmosphere. However, the ratio of the areas of the cross-sections of the expulsion channel 31 and of the third opening 123 is such that the depression is generated even when in the dosing chamber 12 when the plunger 35 returns to its rest position. Thus, for example, the diameter of the expulsion channel 31 can be of 0.3 mm, which gives an area of the cross-section of 0.7 mm², while the diameter of the third opening 123 is typically around 3 mm, which gives an area of the cross-section of 7 mm², that is a ratio of 1 to 100. However, in a variant, an outlet valve could be considered at the second opening 122 of the dosing chamber, or a stopper at the dispensing aperture 16.

Thus, the invention makes it possible to produce a device without priming.

During the joining of the dispenser 10 to the reservoir 20, the dispensing head 15 and the plunger 35 are in the rest position with respect to the pump body 11. The first opening 121 is therefore open. Thus, when the dispenser 10 is joined to the reservoir 20, the pump body 11 penetrates inside the reservoir 20 and due to this, moves the fluid product contained in the reservoir 20. The volume of moved fluid product corresponds to the volume that the pump body 11 occupies in the reservoir after joining. The fluid product thus moved therefore penetrates inside the dosing chamber 12 via the first opening 121, as illustrated by the arrow in FIGS. 1 and 5. The joining is done in the upright position, the dosing chamber 12 is filled up to the edge of the first opening 121, thus defining the dose to be dispensed. The pump is therefore primed from the joining, and there is no need to perform priming actuations before using the device.

During the first actuation, the user places two fingers on the radial flange 153 formed on the dispensing head 15, and presses with the thumb on the bottom of the reservoir 20. During such an actuation, the reservoir 20 is therefore pushed axially in the direction of the dispensing aperture 16, such that the plunger 35 slides in the dosing chamber 12. The first opening 121 is closed after the initial small stroke d1 and the third opening 123 is closed by the pressure exerted by the fluid product on the stopper member 40 forming a valve. The contents of the dosing chamber 12 is thus dispensed through the second opening 122 towards the dispensing aperture 16.

If the device is a two-dose or a multidose device, when the user releases the pressure on the dispensing head 15 after the first actuation, the spring 13 returns the plunger 35 to its rest position. A depression is thus created in the dosing chamber 12, which opens the third opening 123 to fill the dosing chamber 12 from the reservoir 20 through this third opening 123.

The device is thus ready for a second actuation.

Thus, the first opening 121 ensures the transfer of the first dose into the dosing chamber 12 during the joining, and the third opening 123 ensures the transfer of the second dose (and optionally other additional doses) in the dosing chamber 12 after dispensing of the first dose.

Naturally, variants of embodiment can be considered, without deviating from the scope of the invention, such as defined by the accompanying claims.

1. A fluid product device comprising a dispenser joined to a reservoir which contains fluid product, said dispenser comprising a pump body which contains a dosing chamber, and a dispensing head comprising a dispensing aperture, said dispensing head being axially movable with respect to said pump body between a rest position and an actuation position, a plunger, which is secured to said dispensing head, being designed to slide in said dosing chamber between a rest position and an actuation position, said dosing chamber comprising at least two openings, a first opening for filling said dosing chamber and a second opening connecting said dosing chamber to said dispensing aperture, characterised in that said first opening is made in a cylindrical portion of said pump body in which said plunger slides during actuation, said first opening connecting said dosing chamber to said reservoir when said plunger is in the rest position, and said first opening isolating said dosing chamber from said reservoir when said plunger moves out of its rest position, said dosing chamber being filled through said first opening when said dispenser is joined to said reservoir.

2. The device according to claim 1, wherein a lower edge of said first opening is disposed at rest at a distance from said plunger, such that during actuation, said first opening is closed after an initial stroke corresponding to said distance.

3. The device according to claim 2, wherein said distance forms less than 30% of the total actuation stroke of said plunger, preferably around 20%.

4. The device according to claim 1, wherein said first opening comprises one or more slots formed in the cylindrical portion of said pump body.

5. The device according to claim 1, wherein a hollow insert is disposed in said dispensing head, said hollow insert defining an expulsion channel connecting, during actuation, said dosing chamber to said dispensing aperture, said hollow insert comprising said plunger.

6. The device according to claim 5, wherein said piston is formed by a seal, in particular an O-ring, joined to said hollow insert.

7. The device according to claim 1, wherein said reservoir only contains a single dose of fluid product, dispensed in a single actuation.

8. The device according to claim 1, wherein said reservoir contains several doses of fluid product dispensed in several successive actuations.

9. The device according to claim 8, wherein said reservoir contains two doses of fluid product dispensed in two successive actuations.

10. The device according to claim 8, wherein said dosing chamber comprises a third opening provided at the lower axial end of said pump body, said third opening comprising a stopper member, such as a ball, forming an inlet valve for said dosing chamber for filling it after each actuation.

11. A method for priming a device for dispensing a fluid product, comprising the following steps:
providing a reservoir which contains at least one dose of fluid product;
providing a dispenser comprising a pump body which contains a dosing chamber, and a dispensing head comprising a dispensing aperture, said dispensing head being axially movable with respect to said pump body between a rest position and an actuation position, a plunger, which is secured to said dispensing head, being designed to slide in said dosing chamber between a rest position and an actuation position, said dosing chamber comprising at least two openings, a first opening for filling said dosing chamber and a second opening connecting said dosing chamber to said dispensing aperture, said first opening being made in a cylindrical portion of said pump body in which said plunger slides during actuation, said first opening connecting said dosing chamber to said reservoir when said piston is in the rest position, and said first opening isolating said dosing chamber from said reservoir when said plunger is moved to its actuation position,
said method comprises the step of joining said dispenser to said reservoir, by inserting said pump body in said reservoir with said plunger in the rest position, this insertion moving the fluid product contained in said reservoir, said moved fluid product penetrating inside said dosing chamber through said first opening, to thus prime the device.
